(19) United States
(12) Patent Application Publication
(10) Pub. No.: US 2010/0161032 A1
(43) Pub. Date: Jun. 24, 2010

(54) BIOLOGICALLY ENGINEERED STENT

(76) Inventor: Francisco Avellanet, Westport, CT (US)

Correspondence Address: Margaret J. McLaren, P.A.;McLaren Legal Services, 6500 S.W. 133rd Drive, Miami, FL, 33156, US

(21) Appl. No.: 12/733,138

(22) PCT Filed: Aug. 14, 2008
(86) PCT No.: PCT/US08/73145

§ 371 Date: Feb. 12, 2010

Related U.S. Application Data
(60) Provisional application No. 60/956,046, filed on Aug. 15, 2007.

(51) Int. Cl. A61F 2/82; A61K 31/7088

(52) U.S. Cl. 623/1.15; 623/1.42; 514/44 R

(57) ABSTRACT

Biologically engineered stents are provided, some having novel double-walled and hybrid composition constructions that are suitable for multi-drug delivery. Some embodiments of biologically engineered stents (BES) in accordance with the invention can deliver drugs in the form of gene therapy vectors to cells in the walls of stented vessels, thereby promoting local production of therapeutic factors that attract and enhance the formation of endothelium in the stented vessel. Other embodiments of BES include xenografts, allografts or isografts comprising sleeve-like natural matrices derived from vessels of animal and human subjects including postmortem human donors.

### CROSS-REFERENCE TO RELATED APPLICATIONS

The present application claims priority to U.S. Provisional Patent Application No. 60/956,046 entitled Biologically Engineered Stent, filed Aug. 15, 2007, the disclosure of which is hereby incorporated by reference in its entirety.

### FIELD OF THE INVENTION

The invention generally relates to stents, which are medical devices that are used to open and maintain patency in vessels of the body, for example to maintain blood flow through diseased blood vessels. More specifically, the invention relates to biologically engineered stents that are useful for localized delivery of therapeutic drugs, molecules and cells to the walls of damaged vessels, following implantation of the stent in the vessel.

### BACKGROUND

Today more than one million balloon angioplasties are conducted annually. In many cases, stents are implanted in an effort to maintain patency of the vessel after angioplasty. Two types of stents are presently approved by the FDA, i.e., the bare metal stent (BMS) and the drug-eluting stent (DES). DES rely on drugs to inhibit the inflammation and scarring caused by the stent pressing against the artery wall. DES release potent cytostatic and cytotoxic compounds to inhibit neointimal growth.

Following a revascularization procedure (e.g., percutaneous transluminal coronary angioplasty, PTCA), narrowing of a coronary artery (restenosis) can and does occur. In time, so much scar tissue can develop that the flow of blood through the blood vessel is prevented, resulting in a condition known as restenosis.

Drug-coated stents have been found to prevent the aggressive growth of scar tissue on the surface of the stent. Therefore, the majority of stents being implanted today are of the drug-eluting type. Several companies market drug-eluting stents that diminish the tendency of stented arteries to restenose. Among these companies are Boston Scientific, with a placlitaxel drug-coated stent called Taxus™ and Johnson & Johnson, having a sirolimus drug-coated stent called Cypher™

Drug-eluding metal stents are typically deployed via catheter. The stent is mounted over a balloon catheter and once the diseased location is reached, the balloon is inflated and then deployed by inflating the balloon and stretching the stent. The coated stent is left in place and over a period of hours or days, the drug begins to elute from the stent into the wall of the artery.

Despite the improvements offered by DES, unfortunately, recently it has become apparent that a major limitation of existing DES is their tendency to increase the risk of life-threatening blood clots that form on the surface of the stent, even years after the stent has been implanted. It has been proposed that this serious side effect of DES, known as “late stent thrombosis” could be due either to the materials used to coat the DES, or to the drugs themselves.

Clearly, there exists an urgent unmet need for improved stents that can maintain long-term patency of such vessels without increasing the risk of serious complications such as late stent thrombosis and heart attack.

### SUMMARY OF THE INVENTION

The invention provides in one aspect drug-eluting stents having novel double-walled and hybrid composition constructions suitable for multi-drug delivery, including embodiments termed “biologically engineered stents (BES).” One preferred embodiment of a BES can deliver drugs in the form of gene therapy vectors to cells in the walls of stented vessels, causing the cells to produce therapeutic factors that promote the formation of endothelium in the vessel. Another preferred embodiment of a BES in accordance with the invention is a stent comprising a biologically derived sheath of matrix prepared from a biological conduit such as an artery, vein, or lymphatic vessel from a subject.

Accordingly, and in one aspect, the invention provides a double-walled stent comprising an outer stent fabricated from a first core material and an inner stent fabricated from a second core material, wherein the inner stent is disposed within the outer stent. The core material of the outer stent and the core material of the inner stent can be made of the same type of material, such as a metal or a polymer. Alternatively, the core material of the outer stent can be a metal and the core material of the inner stent can be a suitable polymer, or vice versa.

Double-walled stents in accordance with the invention can further comprise a drug-containing coating disposed on a surface of the core material of one or both of the stents. In some embodiments, the drug-containing coating is disposed only on a surface of the inner stent, for example only on an inner surface of the inner stent, or on all surfaces of the inner stent.

Some preferred embodiments of the double-walled stents further comprise one or more polymer layers disposed in the space between the outer and inner stents.

The polymer layer can comprise one or more drugs selected from an anti-proliferative drug, an anticoagulant drug, and a chemotactic drug. The polymer layer can comprise a plurality of layers, each of which comprises a different drug.

Another preferred embodiment of a stent in accordance with the invention is a hybrid stent comprising a mid-section and two or more end sections adjoined thereto, wherein the mid-section is fabricated from a core material that is a metal and the end sections are fabricated from a core material that is a polymer.

The mid-section can be fabricated from a balloon-expandable or self-expanding metal. The end sections can be fabricated from a bioabsorbable or biodegradable polymer. The hybrid stents can further comprise a coating or polymer layer containing one or more drugs, selected from an anti-proliferative drug, an anticoagulant drug, and a chemotactic drug.

Another aspect of the invention is a biologically engineered stent (BES) for promoting the formation of vascular endothelium in a blood vessel by gene therapy. The stent comprises a core and one or more drug-containing layers.

In one embodiment, the drug-containing layer includes a gene therapy vector that comprises a nucleic acid sequence that encodes one or more factors that promote endothelial cell chemotaxis or proliferation. A preferred vector in accordance with the invention is an adeno-associated virus (AAV) vector.

In some embodiments, the drug-containing layer further comprises a vector that includes a nucleic acid sequence that encodes an anti-inflammatory agent.

Yet another aspect of the invention is a biologically engineered stent (BES) for placement in a host blood vessel of a subject in need thereof, comprising:

a core material and a biological matrix derived from a tissue of a mammal. The matrix is configured on the stent so as to oppose the intima of the host blood vessel upon implantation of the stent into the subject's vessel.

In some preferred embodiments, the matrix is derived from a donor vessel such as a vein including an umbilical vein, an artery, an arteriole, or a lymphatic vessel.

The matrix of the BES can be prepared from a donor vessel that has been denuded of its endogenous cellular components (“decellularized”) to provide a substrate suitable for the attachment of human stem cells such as cells of the endothelial cell lineage, including endothelial progenitor cells (EPC) and their progeny.

Some embodiments of BES in accordance with the invention include attached stem cells of the endothelial cell lineage that have been previously cultivated in vitro on a decellularized vessel matrix. In one preferred embodiment, the donor vessel from which the matrix is derived is an allograft prepared from a postmortem human vessel.

Other embodiments of BES include vessel-derived matrices that are designed to be implanted with the matrices in an acellular form. Upon implantation, the biological matrix becomes populated with the host's endogenous EPCs and their progeny.

In other embodiments, the donor vessel is an isograft obtained from the body of the subject in need of the stent. The isograft can be cultured for attachment of stem cells, including stem cells derived from the patient.

Stents of the invention including biological matrices can further comprise one or more drug-containing layers for attracting, differentiating, or proliferating cells of endothelial lineage or other purposes as described above.

These and other aspects and advantages of the invention are further discussed below.

### BRIEF DESCRIPTION OF THE DRAWINGS

FIG. 1 is a schematic diagram showing a cross-sectional view of a double-walled drug-eluting stent 100, in accordance with an embodiment of the invention.

FIG. 2 is a schematic diagram depicting a cross-sectional view of a multi-drug stent 200, in accordance with an embodiment of the invention.

FIGS. 3A-C are three schematic diagrams depicting an embodiment of a hybrid multi-drug stent 300 of the invention. FIG. 3A is a sectional view through the longitudinal axis of stent 300 and FIG. 3B is a perspective view of stent 300.

FIG. 3C depicts a cross sectional view along the long axis of an artery wall having damaged areas 305 flanked by normal areas 310 on either side, showing placement of a stent 300 in the artery lumen.

FIG. 4 is a perspective view of a multi-part hybrid stent 400, in accordance with an embodiment of the invention.

FIG. 5 is a perspective view of a multi-part hybrid stent 500, in accordance with an embodiment of the invention.

### DETAILED DESCRIPTION OF THE INVENTION

The present invention provides novel drug-eluting stents (DES), designed for improved performance over existing stents. In various embodiments the improved stents feature one or more drugs for reducing restenosis and thrombosis, and for encouraging the development of a layer of endothelium over the stent after placement in a subject's blood vessel.

As discussed above, coronary artery re-occlusion in humans still remains a drawback of percutaneous coronary interventions, even in the era of drug-eluding stents (DES). The working principle of a DES involves the delivery of controlled amounts of anti-proliferative agents at the local level, with the aim of suppressing neontimal proliferation, the main cause of lumen re-narrowing after a stent has been implanted. At present, several DES platforms have been developed and evaluated for clinical use. With regard to stent type, the differences between them include: (1) metal used to fabricate the stent; (2) anti-proliferative drug used; (3) type of polymers employed for drug storage; and (4) modification of drug release kinetics.

Although the mid-term efficacy of DES has been well established, there is an ongoing debate as to the potential for increase of late stent thrombosis, particularly after discontinuation of thieopyridine therapy, as well as delayed onset of restenosis or “catch-up phenomenon.” Based on human pathological data, investigators have linked the above-mentioned concerns to the presence of polymers in DES, which may have pro-inflamatory and prothrombogenic potential, and may induce a hypersensitivity reaction. The stents described herein are designed to address and overcome at least some of the limitations of presently available DES.

### DEFINITIONS

The term “stent” as used herein refers to a type of mechanical scaffolding used to hold open a blood vessel or other tubular anatomical structure such as a previously occluded blood vessel, in order to restore patency and blood flow in the case of a blood vessel. Several types of stents and stent materials are known, including bioabsorbable polymer stents, balloon-expandable stents and self-expanding stents. A “balloon-expandable stent” comprises a metal tube, typically fabricated from stainless steel, chromium-cobalt alloy or other alloys, which is perforated in a pattern using a laser beam to add flexibility to the tube. To deliver a balloon-expandable stent, a surgeon places the stent over a balloon catheter, locates the catheter at the preselected target site in a damaged blood vessel, and expands the stent by applying pressure to the balloon catheter. A “self-expanding” stent is a type of wire form typically made from Nitinol (nickel-titanium alloy) which has “memory.” This type of stent is placed over a catheter with a sleeve over the stent to hold it in a closed position. Once the target site is reached, the sleeve is removed and the stent springs open (self-expands).

The term “biologically engineered stent” (“BES”) is meant to refer to a stent that incorporates a combination of sciences and technologies, e.g., biotechnology or medical science with biomedical engineering technology, all into one safe and efficacious medical device. A BES in accordance with the invention is a stent fabricated from a man-made material such as a metal and/or a polymer that further incorporates one or more “biological” components, i.e., components obtained from or derived from natural biological sources. Depending upon the application, a “biological component” of a BES, as the term is used herein, can encompass one or more of a wide range of components derived from living organisms, including, but not limited to: sleeves of biological materials derived from naturally occurring expandable “biological conduits” such as arteries, veins, and lymphatic vessels that are used, e.g. to cover one or more surfaces of a stent; stem cells that are incorporated into the stent before implantation; recombinant nucleic acids such as gene therapy vectors designed to locally deliver desired therapeutic genes to cells in the vicinity of a patient's stented blood vessel; proteins such as antibodies designed to attract endothelial progenitor cells (EPC) from the patient's circulation to encourage the establishment of an endothelial layer over the surface of the stent or various growth factors selected to promote the proliferation and differentiation of EPC into endothelial cells. One primary objective of various embodiments of BES in accordance with the invention, as further described below, is to turn into reality the promise of stem cell biology when integrated with a medical device, as a foundation for the treatment and cure of restenosis and late stent thrombosis, as well as treatment of a wide range of other medical conditions.

As used herein, the term “anti-proliferative drug” is meant to include any compound that is effective for arresting or delaying the proliferation of cells in the walls of blood vessels, such as the abnormal neointimal proliferation that leads to restenosis following placement of a stent in a damaged coronary artery of a human subject. Suitable anti-proliferative drugs known in the art include, but are not limited to, cytostatic drugs such as sirolimus and cytotoxic drugs such as paclitaxel.

The term “anticoagulant drug,” as used herein refers to any compound that is useful for preventing the formation of blood clots, including but not limited to antithrombotic, anti-platelet and thrombolytic classes of drugs, including but not limited to warafin, heparin, clopidogrel, dipyridamole, enoxaparin, ardeparin, dalteparin, ticlopidine, danaparoid, tinzaparin, aspirin, and thrombin inhibitors.

The term “chemotactic drug,” as used herein, is meant to broadly refer to any suitable compound having the property of promoting the attraction, homing and attachment of endothelial cells or their precursors (endothelial progenitor cells, EPCs) to a medical device such as a stent, particularly a stent that is implanted in vivo in a blood vessel. In various embodiments of the DES/BES of the invention, chemotactic drugs are either small molecules or biologics (proteins including antibodies, peptides, and nucleic acids including gene therapy vectors comprising promoter and regulatory sequences and nucleotide sequences encoding chemotactic factors).

The terms “allograft,” “homograft,” or “allogeneic graft” as used herein refer to an organ or tissue from one individual that is used for transplantation into another of the same species with a different genotype. For example, a transplant from one human to another who is not an identical twin is an allograft. Allografts account for many human transplants, including those from cadaveric, living related, and living unrelated donors. By contrast, an “isograft,” as the term is used herein, refers to a graft of tissue that is obtained from a donor genetically identical to the recipient. One example of an isograft in accordance with the invention is a vessel that is obtained from a patient for the purpose of combining the vessel, after processing, with a stent to be implanted into the patient. An allograft in accordance with the invention could include, for example, a suitably processed vessel derived from a human cadaver and combined with a stent for use in a patient. Alternatively, an allograft for use in a stent could be derived from a processed human umbilical cord vessel such as a vein.

### Construction of Drug-Coated Stents

Stents in accordance with the invention include one or more core materials that make up the wall(s) of the stent and one or more coating layers that may contain drugs.

The coating layer of a stent in accordance with the present invention can be applied uniformly around the surface of the core material. Alternatively, in some embodiments of the invention, the coating layer(s) is applied to only a portion of the core material, for example by keeping the outer surface of a metal stent free of polymer coatings and applying a drug-containing polymer layer only to the inside wall of the stent. This configuration permits an uncoated outside surface of the core material of the stent (such as a metal wire) to be in contact the intima of the blood vessel when implanted, with the drug-eluting surface being on an inside surface of the stent. In some applications this arrangement may be advantageous and preferred, for example to minimize the likelihood of toxicity, based on recent evidence that the cause of blood clots in patients with implanted DES is long-term contact of the drug-containing polymer with the intima. For example, recent autopsy data presented by a leading cardiovascular pathologist identified the five primary causes of late stent thrombosis as stent mal-apposition, stent struts embedded in a necrotic core, hypersensitivity reaction to the implant, discontinuation of anti-platelet therapy, and ostial or bifurcation stenting (R. Vermani, Trans-catheter Therapeutics Annual Meeting, October 206, Washington, D.C.)

Some preferred embodiments of metal stents in accordance with the invention are made of a permanent metal as described herein, which is additionally coated with a magnesium layer. The magnesium plating is interposed between the permanent metal and the coating material comprising the drug. After a period of time following placement of the stent in the vessel, the magnesium coating dissolves, leaving the metal stent in place as a permanent scaffold.

The coating material used to store and release the drug can be a biodegradable polymer or a bioabsorbable polymer. Suitable polymers include polyethylene, polypropylene, polymethacrylate or aromatic polymers with benzene rings such as polystyrene, polycarbonate or acrylate epoxies. Alternatively, the polymers can be a porous type of material such as ePTFE, Gore-Tex, or a non-porous material such as a thermoplastic elastomer, including polyurethane or silicone rubber. Polymers particularly suitable for seating against the inner surface of a stent can be ePTFE, Gore-Tex or a similar porous and stretchable polymeric material.

Some embodiments of stents in accordance with the invention are configured to deliver drugs in the form of biologics including gene therapy vectors such as AAV vectors. The vectors can include nucleic acid sequences encoding therapeutic proteins or inhibitory RNA molecules, for example. Biocompatible delivery matrices and vehicles have been developed for sustained release of proteins, drugs, and nucleic acids including AAVs and RNAi from surface coatings on metal stents, or from embedded microporous capsules or microspheres within these coatings. Suitable materials include, but are not limited to negatively charged gelatin-polyglutamic acid hydrogel films; positively charged gelatin-polylysine hydrogel films, polylactic-co-glycolide (PLGA) microspheres of various sizes; gelatin-heparin microspheres; photosensitive gelatin-nitroinnamate hydrogel scaffolds; and gelatin-heparin (negatively charged) crosslined polymers. Such materials and their uses with biologics are described, e.g. in Zheng Y et al., Advanced Functional Materials 2001, Vol. 11, No. 1, 37-40; Gattas-Asfura K M et Biomacromolecules 2005, 6:1503-1509; Andrepoulos F M et al. Biomaterials 2006, 27:2468-2476; and Layman H et al., Biomaterials 2007, 28:2646-2654.

In some instances, it may be advantageous to modify the surface of stents (particularly metal stents) to promote and improve adherence of desired additions to the stent, such as polymer coatings, drugs, nanoparticles, or cells. The surface can be modified by creating small or microscopic “nests” for deposition on the stent surface. For example, an anti-proliferative or anticoagulant drug can be deposited on the roughened surface of a metal stent, and later coated with a polymer, pyralene or a similar material so as to seal the drug and allow controlled release of the drug(s) over time.

Many suitable methods can be used to modify the inner or outer surfaces of metal stents, including but not limited to: various mechanical approaches (e.g., shot peening, sanding, sand blasting or grinding, knurling-straight or diamond finish, thread-rolling, cold rolling, drawing through a die, or swaging); chemical techniques (e.g., acid etching, electropolishing or electroplating, oxidizing, or plating, such as nickel plating or anodizing); radiation (laser peening, X-raying, electron beam); vacuum deposition of other metals; welding, such as TIG or plasma welding; coating, including porous powder, metal or ceramic; and heat treating to change the grain structure of the metal.

Stents in accordance with the invention can be produced using conventional methods of stent fabrication, which are known in the art. One common practice involves cutting out “windows” on thin-walled metal tubing using a laser beam. Alternatively, the stents can be produced by cutting metal squares or rectangles with their respective cut-outs, and optionally coating them on one or both sides and loading them with a drug. The uncoated or coated squares are then wrapped around a mandrel with ends abutting, and welded to form a tube.

### Double-walled Stents

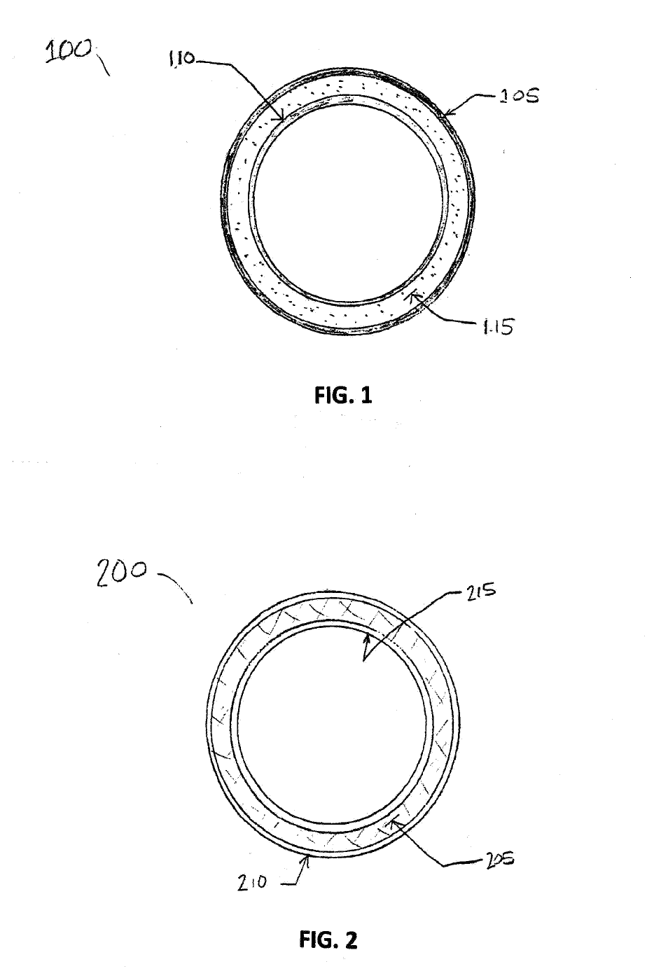

Some embodiments of stents in accordance with the invention are configured as double-walled stents. Referring to FIG. 1, there is shown one configuration of a drug-eluting stent with double walls, or a “stent-within-a-stent” 100. Positioned between the outer stent 105 and the inner stent 110 is a non-metallic layer 115 comprising a polymer or other biocompatible material suitable for drug-loading.

The walls of the inner and outer stents 105 and 110 can be fabricated from any suitable metal or polymer. Two metal stents can be fabricated using the same or dissimilar metals. For example, ensuring that no galvanic corrosion is produced, the inner stent 110 can be made of a malleable, non-thrombogenic material such as platinum, tantalum, magnesium, cobalt-chromium, gold, or an alloy thereof, and the outer stent 105 can be made of a more rigid metal such as stainless steel. The reverse combination can also be produced, e.g., a stainless steel stent can be used on the inside and a stent made of a softer metal can be on the outside. The construction of stent walls having two different diameters (tube within a tube) is designed to allow a balloon catheter to expand the composite stents radially, without substantially building up the profile of the stent.

Some preferred embodiments of double-walled stents in accordance with the invention are composite structural stents having the inner and outer walls fabricated from metal, with a softer, low strength core material placed between the metal layers. In one preferred embodiment, the double-walled stent is fabricated using two self-expanding Nitinol wire stents, with one or more drug-loaded polymer layers 115 sandwiched between the inner and outer wire stents 105, 110. Another configuration of a composite double metal stent can be made by combining a wire stent with a tubing stent. Both the wire stent and the tubing stent can be balloon-expandable or self-expanding (Nitinol wire), or a combination of the two.

Once the assembly of inner and outer metal stents and the core material (such as a drug-containing polymer layer) is complete, the metal stents can be welded, crimped or bonded at the ends or at one or more locations along their length to prevent axial displacement of the stents relative to one another.

Another embodiment of a double-walled stent in accordance with the invention is a stent that combines an outer metal layer with an inner polymer or thermoplastic elastomer layer. The polymer or thermoplastic material can be the type that can be cross-linked (leading to modifications of the polymers) by chemical reaction or with UV light, nuclear radiation, radio-frequency, or any other safe and efficacious energy source that can be used in vivo, i.e., from the inside of a blood vessel during surgery. Alternatively, the cross-linking can be done from the exterior of the human body using a known safe energy source such as ionizing radiation used in food irradiation and sterilization of healthcare products, X-rays, radio frequency, magnetic resonance, magnetic field, electron beam, gamma radiation and the like.

The drug-loaded polymer positioned between the two metal layers can be of a type that is biodegradable, or of a more permanent type. Either type of polymer used can be curable by ultraviolet light. In the latter case, the biodegradable polymer is a polymer that can harden, cure, cross-link, or polymerize when contacted with photons emitted by ultraviolet (UV) light such that when the stent is being inserted into the human vasculature, the polymer is soft. Once the stent is deployed at the chosen site, a UV light is inserted via a catheter into the blood vessel and light is applied to the polymer on the stent, causing it to harden. The UV light can be transmitted through one or more optical fibers attached to a guide wire or a catheter.

### Multi-Drug Eluting Stents

Some embodiments of stents in accordance with the present invention are capable of delivering two or more drugs, such as an anti-proliferative drug and an anticoagulant, and/or chemotactic drug.

One embodiment of a multi-drug stent 200 in accordance with the invention is schematically illustrated in FIG. 2. The stent 200 comprises a single-walled metal or polymer stent 205 that is coated on its outer surface with a layer 210 comprising a first drug and coated on its inner surface with a layer 215 comprising a second drug.

Another preferred embodiment of a dual drug-delivering stent is a double-walled stent that is coated on one or more surfaces of the outer stent with a first class of drug, e.g., an anti-proliferative drug. A drug of a second class, e.g., an anticoagulant drug, is further included within a polymer layer sandwiched between the inner and outer stents.

In yet another embodiment of a dual or triple-drug delivering stent, in this case designed to leave the surface of the outer stent facing the intima uncoated, the polymer included in the space between the inner and outer stents comprises two or more layers, each containing a different drug. The drugs can be advantageously arranged in layers that optimize the different kinetics of the drugs, or the desired timing of delivery of the drug to the blood vessel.

Delivery of the drugs from the polymers can be controlled by the choice of polymers. For example, biodegradable polymers curable by UV light can be formulated in such a way that different hardness, and hence drug delivery kinetics, can be achieved based on the frequency, light intensity, and time that the polymer is exposed to UV light of various wavelengths (UVA, UVB or UVC).

Many desirable combinations of drugs that can be delivered by the multi-drug delivering stents of the invention will be readily apparent to those of skill in the art. A non-limiting example of a dual drug delivering stent in accordance with the invention comprises a biodegradable polymer layer positioned in the space between the walls of the inner and outer stents that includes an outermost layer loaded with an anti-proliferative drug, and an inner layer loaded with an anticoagulant drug. An exemplary triple drug-delivering stent comprises three layers of biodegradable polymer containing, e.g., from outermost to innermost, an anti-proliferative drug, an anticoagulant drug, and a chemotactic drug to promote endothelialization of the stent.

Due to its position closest to contact with the patient's body, the drug that is loaded in the outermost layer of a biodegradable polymer on the stent will dissolve from the polymer first, and accordingly will be delivered to the blood vessel wall before a drug contained in a deeper layer on the stent. Loading an anti-proliferative drug in the outermost layer provides the initial benefit of regulating aggressive scar tissue formation that can occur soon after implantation of the stent. Once the outermost layer of biodegradable polymer is dissolved, the next underlying layer containing the anticoagulant drug is released to the blood vessel. Preferably, the anticoagulant drug is formulated in a polymer that allows a long release life from the polymer, thereby providing protection against formation of blood clots over a long period of time following implantation of the stent into the patient.

Another embodiment of a double-walled multi-drug stent in accordance with the invention includes a multi-polymer layer positioned between the walls of the inner and outer stents, wherein the polymer layer comprises an outer layer of biodegradable polymer and an inner layer of a non-degradable polymer. The outer biodegradable layer can be loaded, e.g., with an anti-proliferative drug that will be released as the biodegradable layer dissolves, and the non-degradable polymer can be loaded with an anticoagulant drug.

### Hybrid Drug-Eluting Stents

The invention further provides multi-drug delivering stents that are termed “hybrid” drug-eluting stents owing to their construction from several adjoined sections made of differing materials and/or coatings, aligned along the longitudinal axis of the stent. Those of skill in the art will appreciate that many variations of hybrid stents comprising several different construction materials and drugs can be envisioned.

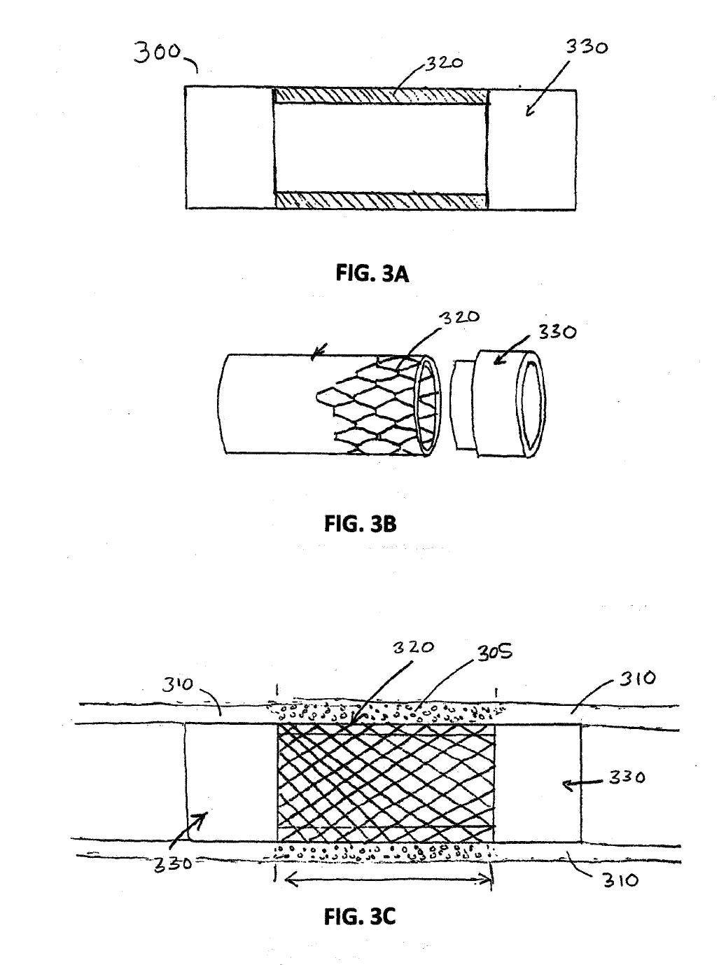

FIGS. 3A-C illustrate several views of a hybrid stent 300 in accordance with the invention that comprises two sections 330 at either end of the stent 300 that are made from a permanent or a biodegradable polymer, and a balloon-expandable mid-section 320 made of coated or bare metal, all sections being attached together integrally. In the embodiment 300 shown, end sections 330 are configured as inserts that fit into the distal ends of mid-section 320, but any suitable means of attaching the end- and mid-sections can be used.

In one configuration of the stent 300, the mid-section 320 is coated with one or more layers comprising an anti-proliferative drug, and the two end sections 330 of the stent are coated with an anti-coagulating drug. Any or all of the drug-containing layers can be conditionally degradable with a drug that triggers degradation, in order to allow a second or third layer to activate the release of a desired bioactive compound.

FIG. 3C is a schematic diagram showing placement of a hybrid stent 300 after implantation into a blood vessel in need of stenting. Referring to FIG. 3C, it is seen that the distribution and combination of materials in hybrid stents such as stent 300 of the invention provides for a multi-drug stent comprising multiple adjacent sections (three shown in FIG. 3) in which the mid-section 320 of the stent 300 can be located adjacent to the damaged area 305 within the vessel wall, and the two end sections 330 can extend beyond the area of the lesion 305, to overlie the more normal areas 310 of the blood vessel wall outside of the damaged area.

Any suitable drug or combination of drugs can be delivered by each section of the hybrid stent 300. One preferred embodiment is a hybrid DES in which the two end sections 330 are loaded with an anti-proliferative drug and the mid-section 320 includes an anti-coagulant drug, for example included in a coating over a permanent polymer. In another preferred embodiment, the positioning of the two classes of drugs is reversed within the sections of the hybrid stent 300.

Yet another embodiment of hybrid stent 300 is a biologically engineered variation of the stent, or a “biologically engineered stent,” (BES), further described below, in which one or more of the drugs contained in the stent is a biologic, such as a gene therapy vector, that can be eluted from one or more sections of the hybrid stent. Upon release from the stent, the vectors are available to deliver therapeutic genes to cells in the walls of the vessel, either in the area of the lesion 320 or in the adjacent areas of the vessel wall 310 having more normal structure and presumably function.

The materials used to fabricate the sections of the hybrid stent 300 are not limited to combinations of metal and polymer materials; for example a hybrid DES in accordance with the invention can also be fabricated from only metal components, or only polymer components. Hybrid multi-section DES in accordance with the invention can also encompass stents in which each section is made using UV light curable polymer, and each section is loaded with a different drug to either provide anti-proliferation or anti-coagulant therapeutics, as well as providing different sections with different degrees of hardness hardening with UV light as described above.

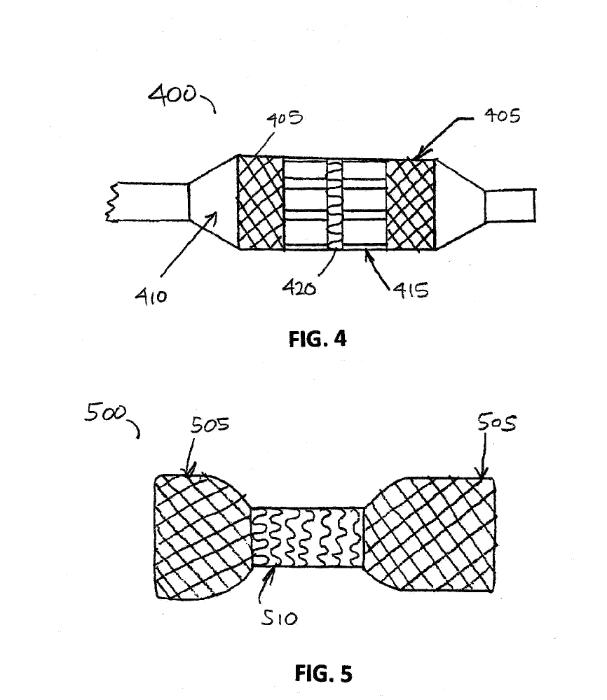

Another preferred embodiment of a hybrid DES in accordance with the invention is hybrid stent 400, illustrated schematically in FIG. 4. Stent 400 is configured with the objective of providing uniform drug dosing, high radial strength, and thus and superior scaffolding, and avoidance of separation of the stent 400 from the vessel wall. Hybrid stent 400 is a multi-section DES comprising three balloon expandable or self-expanding sections, two extreme ones 405 and a middle one 415 consisting of a ring 420 with two or more flat wires, cables, or thins struts 415 between the sections 405.

In an alternate embodiment (FIG. 5), the wires or cables 415 of stent 400 are replaced with a self-expanding or a balloon-expandable coiled wire 510, as illustrated in hybrid stent 500 having self-expanding end sections 505, shown in FIG. 5. All sections of hybrid stents 400 and 500 can be drug-coated as described supra.

### Biologically Engineered Stents (BES)

As mentioned supra, BES are stents in accordance with the instant invention that incorporate one or more biological materials into the stent. An important principle that underlies a BES of the invention is a design that incorporates biological elements aimed to promote the formation of endothelium in a damaged blood vessel into which the stent is implanted, thereby promoting normal architecture in the underlying vessel and preventing restenosis.

An appreciation of the advantages of a BES in accordance with the present invention will be gained from a brief review of the anatomical structure of a blood vessel such as an artery, and in particular an awareness of recent biological findings relating to endothelial cells that form the smooth lining of blood vessels including arteries.

As mentioned previously, coronary arteries of the heart, when diseased, are common sites for surgical implantation of stents. From outermost to innermost, the walls of these arteries are made up of several layers known as the adventitia, media, and intima. The channel surrounded by the wall of an artery or other blood vessel is termed the lumen. Lining the surface of the vessel that faces the lumen is the innermost layer of the intima, known as the endothelium.

The endothelium is made up of flattened, tightly connected cells known as endothelial cells that have a cobblestone appearance when viewed en face. The endothelial layer of a blood vessel is bathed by the blood, and its cells are constantly subjected to the turbulence of blood flow and other stresses. Endothelial cells that are located at bends in arteries, or at sites where arteries branch (bifurcations), are subjected to greater-than-average stress, as evidenced by the frequency of vessel damage at such sites, including accumulation of atherosclerotic plaques that can narrow or completely occlude the artery.

As discussed above, increased risk of late stent thrombosis, i.e., blood clotting at the site of stent implantation, has been associated with stents that elute drugs such as paclitaxel and sirolimus. Studies have shown that blood platelets, which aggregate inside blood vessels to form dangerous blood clots, are less likely to adhere to the walls of blood vessels that are covered with an intact endothelium, as compared with damaged or unhealed areas of a vessel wall that are denuded of endothelium. Additionally, the presence of an intact endothelium is known to inhibit the uncontrolled proliferation of smooth muscle cells of the blood vessel media that can contribute to restenosis. For these reasons, it is highly desirable for an implanted stent to be covered by an intact layer of endothelium.

Recently it has been established that the human body has a natural repair process for replacing lost or damaged endothelial cells in blood vessels (Asahara et al. 1996). Cells known as “endothelial progenitor cells” (“EPCs”) are bone marrow-derived stem cells that circulate in the bloodstream and have the ability to home to blood vessel walls and differentiate into mature, functional endothelial cells that integrate into the endothelium.

Based on the above observations and principles, BES in accordance with the invention are designed to attract circulating EPCs and/or to promote the formation of an endothelial layer over the area of stent implantation. More specifically, upon implantation into a blood vessel, drug-eluting embodiments of BES in accordance with the invention are designed to release drugs that promote the growth of an intact layer of endothelium over the stent and the adjacent vessel wall underlying the struts of the stent, thereby reducing the likelihood of stent-related complications including both restenosis and thrombosis.

### Biologically Engineered Stent for Gene Therapy of Blood Vessels

One particularly preferred embodiment of a BES in accordance with the invention addresses the above-described objective by providing a stent that permits localized delivery of effective amounts of therapeutic genes to cells in the walls of the blood vessel in the vicinity of the implanted stent.

Accordingly, one important aspect of the invention is a biologically engineered gene therapy stent for promoting formation of vascular endothelium in a blood vessel. The stent comprises a core and one or more coating layers. Materials suitable for fabricating the cores and coating materials of the BES are essentially as described above. Any one of the multi-drug and multi-section hybrid stents described above can be configured as a BES in accordance with the invention by adding to the drug-containing coating or polymer layers of the stent one or more biologics that can promote the attraction, proliferation and differentiation of cells in the endothelial cell lineage, causing them to home to the area of the stent and to form a functional endothelial layer over the stent surface.

In one preferred embodiment, the drug contained in the BES is a chemotactic drug in the form of a gene therapy vector that comprises a nucleotide sequence that encodes one or more factors that promote endothelial cell chemotaxis (chemoattraction) and/or proliferation. The factors encoded by the nucleotide sequence can include at least one of SDF-1, VEGF, MCP-1 and HGF.

Vectors suitable for gene therapy are well known in the art. A preferred vector in accordance with the invention is a recombinant adeno-associated virus (rAAV) vector. The rAAV vector can be of any known rAAV serotype.

In some embodiments, the coating layer of a BES intended for gene therapy further comprises an anti-inflammatory drug, which may also be in the form of a gene therapy vector that includes a nucleic acid sequence that encodes an anti-inflammatory agent. Anti-inflammatory agents can include endothelial and inducible nitric oxide synthetases (ENOS and iNOS); peroxisome proliferator-activated receptors alpha and gamma (PPAR α and γ); adiponectin; apolipoprotein M (ApoM); apolipoprotein A-1 mimetic peptides; NK kappa B siRNA; superoxide dismutase; thioredoxin; and HLA-G.

The expression of the anti-inflammatory agent or other drug in the gene therapy vector is controlled by one or more promoter sequences. In some embodiments, the expression of the therapeutic drug sequence by the vector is conditionally inducible, for example using a “molecular switch” that responds to local physiological conditions such as anoxia. Suitable molecular switches for mammalian gene expression are described, e.g., in U.S. Pat. No. 6,893,867.

### BES Comprising Matrix Derived from a Biological Source

Yet another embodiment of a BES in accordance with the invention is a novel stent that incorporates a matrix that is derived from a natural biological source, e.g., from a blood vessel such as a vein. The vessel matrix can serve many functions, including but not limited to providing a substrate for attachment of cells such as stem cells and endothelial cells. Without intending to be bound by any particular theory, it is believed that a naturally occurring biological matrix derived from healthy biological conduit such as a blood vessel would provide an ideal substrate for attracting, attaching and proliferating stem cells of the endothelial cell lineage and their progeny. Incorporation of a matrix derived from such a vessel would further impart to the stent those intrinsic beneficial properties of the natural biological matrix of a vessel, including generative, regenerative or healing and therapeutic qualities.

In one preferred embodiment, a coronary or vascular stent is covered with a matrix derived from a vessel from a human or other mammal. The “donor” vessel can be a xenograft (for example from a mammal such as a pig or monkey), an allograft (homograft), e.g., a vessel harvested from a human cadaver or from a human umbilical cord, or the vessel can be an isograft extracted from the patient's own body. In the case of an isograft, a process similar to that used in heart bypass surgery is employed to harvest a healthy blood vessel (typically a vein) from a patient in need of a stent. The extracted vessel is then processed as described below for use on the stent.

Allografts can be obtained and processed under the guidance of an accredited tissue bank. Allografts are held in quarantine until microbiological and blood tests are completed. The tests are conducted following strict guidelines required by the Food and Drug Administration and the American Association of Tissue Banks. Required tests include a thorough analysis of infectious diseases such as HIV, hepatitis B and C and syphilis. Established and well known sources of vessel allografts suitable for use in the cardiac field include, for example, Life Net Health (Virginia Beach, Va.) and University of Miami Tissue Bank (Miami, Fla.).

Regardless of the origin of the donor vessel (xenograft, allograft or isograft), in some preferred embodiments of the inventive BES comprising vessel-derived matrices, the donor vessel is decellularized using known methods. The decellularized vascular graft can be stored until use in suitable sterile media such as RPR media or it may be freeze dried and later reconstituted. The purpose of the decellularization process inter alia is to provide a denuded substrate suitable for repopulation with human stem cells, endothelial progenitor cells (EPCs), endothelial cells, or other cell types. It will be apparent to those of skill in the art that the vessel-derived matrix, in combination with a stent, can be used in several ways to achieve the desired objective of covering the stent surface with a healing layer of endothelium.

In one ex vivo approach, a suitably processed denuded vessel of appropriate diameter and thickness is mounted on a metal or bioabsorbable stent and cultured in the presence of endothelial progenitor cells under conditions that promote the attachment, proliferation and differentiation of these cells into an endothelial cell layer that covers one or more surfaces of the stent.

Alternatively, during the culturing process, the denuded vessel can be mounted on a hydrophilic or hydrophobic coated polymer tube, preferably of the bioabsorbable type, to form a subassembly comprising a polymer tube and the vessel. After the culturing is complete, the subassembly, populated with live cells (termed a “vascular graft”) is provided separately from the stent under sterile conditions and can be fitted over a stent at a later time. For example, at the time of surgery, the vascular graft can be fitted over the stent in a sterile field immediately prior to delivery of the stent to the patient's diseased blood vessel.

An alternate approach to in vitro endothelialization of the BES utilizes the environment of the patient's body rather than a tissue culture environment to promote the in vivo formation of an endothelial layer over the stent. According to this method, a BES having a denuded blood vessel-derived matrix is prepared as above, but without the pre-attachment of cells to the vessel matrix in vitro. In this case, the formation of a layer of endothelium on the stent's vessel-derived biological matrix can be allowed to occur naturally in vivo over a period of days or weeks, through the attraction and attachment of EPCs from the patient's circulation. Alternatively or additionally, exogenous stem cells of the endothelial cell lineage or their progeny can be delivered directly to the biological vessel-derived matrix on the stent, for example by means of a cannula, without prior culturing of the stent matrix. Seeding of the stent with cells following its implantation in a patient's vessel is also an option.

As with other stents described above, BES comprising a matrix derived from a natural source such as a vessel can be fabricated from any suitable combination of materials, including various metals and synthetic polymers. Further, BES comprising vessel-derived matrices can be fabricated in any suitable configuration that promotes the association of a layer vascular endothelium with the stent, or otherwise provides the beneficial effects of a drug-eluting stent.

The stent materials and/or the biological vessel-derived matrix can serve as a platform onto which can be loaded microspheres or coatings comprising therapeutic small molecules, proteins, nucleic acids, DNA, or RNA as described above. As discussed, coatings and microspheres of use in the stents of the invention can be designed to perform one or more defined biological functions related to drug delivery, such as gene therapy through controlled gene delivery and targeted controlled release and delivery of DNA, drugs, viruses and other therapeutic agents.

BES comprising matrices derived from natural biological conduits such as de-cellularized veins, as described herein, are envisioned to provide numerous advantages over prior art stents that employ non-biological materials at the interface of the stent with the diseased blood vessel wall in a patient. One important objective of the invention is to avoid or mitigate complications known to be associated with bare metal and drug-eluting coronary stents, including restenosis and acute and late stent thrombosis, by providing stents comprising natural matrix materials derived from blood vessels. These matrices are expected to promote and accelerate healing without introducing deleterious side effects such as neointimal proliferation, inflammation and rejection of the implant caused by the introduction of foreign materials by the stent. It is expected that matrices derived from blood vessels will provide superior biocompatibility and hemocompatibility, as compared with synthetic materials currently in use for this purpose.

Being derived from an abundant natural source, matrices derived from vessels provide the further advantage of are being relatively inexpensive to obtain, requiring no manufacturing, and only minimal processing in order to produce a cell-free biological stent matrix suitable for loading with drugs and/or cells. Vessel-derived biological matrices of different diameters, suitable for various types of applications, are readily available from the wide range of naturally occurring vessels.

Although the BES comprising vessel-derived matrices have been described here with emphasis on particular embodiments of stents suitable for use in diseased coronary arteries, the invention is not so limited. In the field of vascular biology, donor vessels of different sizes can be chosen as appropriate, for use in a wide range of vascular procedures to repair damaged blood vessels. For example, biologically engineered matrices derived from donor vessels could be used to cover coils used to repair aortic aneurysms and in embolization therapy. Additionally, biologically engineered endovascular grafts known as AAA stent grafts can be fabricated using large veins or human umbilical cords. Even more broadly, a wide array of applications is envisioned for stents comprising natural vessel-derived matrices, to provide for patency and healing of tubular structures throughout the body, not only those of the cardiovascular system.

All patents, patent applications and publications cited in this specification are incorporated by reference in their entirety. Such references are also cited as indicative of the skill in the art.

While the invention has been described in connection with what is presently considered to be practical and preferred embodiments thereof, it should be understood that it is not to be limited or restricted to the disclosed embodiments, but rather is intended to cover various modifications, substitutions and combinations within the spirit and scope of the appended claims. In this respect, one should also note that the protection conferred by the claims is determined after their issuance in view of later technical developments and would extend to all legal equivalents.

What is claimed is:

1. A double-walled stent comprising:
an outer stent fabricated from a first core material; and
an inner stent fabricated from a second core material, wherein the inner stent is disposed within the outer stent.

2. The double-walled stent according to claim 1, wherein the first core material and the second core material are the same type of material.

3. The double-walled stent according to claim 2, wherein the material is a metal or a polymer.

4. The double-walled stent according to claim 1, further comprising a drug-containing coating disposed on a surface of the core material of one or both of the stents.

5. The double-walled stent according to claim 4, wherein the drug-containing coating is disposed only on an inner surface of the inner stent.

6. The double-walled stent according to claim 1, wherein the polymer layer comprises a drug selected from the group consisting of an anti-proliferative drug, an anticoagulant drug, and a chemotactic drug.

7. A hybrid stent comprising a mid-section and two or more end sections adjoined thereto, wherein the mid-section is fabricated from a core material that is a metal and the end sections are fabricated from a core material that is a polymer.

8. The hybrid stent according to claim 7, wherein the mid-section is fabricated from a balloon-expandable or self-expanding metal.

9. The hybrid stent according to claim 7, wherein the end section comprises a bioabsorbable or biodegradable polymer.

10. The hybrid stent according to claim 7, further comprising a coating or polymer layer comprising one or more drugs selected from an anti-proliferative drug, an anticoagulant drug, and a chemotactic drug.

11. A biologically engineered stent (BES) for promoting formation of vascular endothelium in a blood vessel comprising:
a core material and a drug-containing layer, wherein the drug-containing layer comprises a vector that includes a nucleic acid sequence encoding a factor that promotes attraction, differentiation, or proliferation of endothelial cells.

12. The BES according to claim 11, wherein the vector is an adeno-associated virus (AAV) vector.

13. The BES according to claim 11, wherein the factor is selected from the group consisting of VEGF, HGF, SDF-1, and MCP-1.

14. The BES according to claim 13, wherein the drug-containing layer further comprises a vector that includes a nucleic acid sequence encoding an anti-inflammatory agent selected from the group consisting of endothelial nitric oxide synthetase (ENOS), inducible nitric oxide synthetase (iNOS), peroxisome proliferator-activated receptor alpha (PPAR α), peroxisome proliferator-activated receptor gamma (PPARγ), adiponectin, apolipoprotein M (ApoM), apolipoprotein A-1 mimetic peptides, NK kappa B siRNA, superoxide dismutase, thioredoxin, and HLA-G.

15. A biologically engineered stent (BES) for placement in a host blood vessel of a subject in need thereof, comprising:
a core material; and
a biological matrix derived from tissue of a mammal, said matrix being configured to oppose the intima of the host blood vessel upon implantation of the stent into said vessel.

16. The BES according to claim 15, wherein the matrix is derived from a donor vessel selected from the group consisting of a vein, an artery, an arteriole, and a lymphatic vessel.

17. The BES according to claim 16, wherein the matrix is prepared from a donor vessel that has been decellularized.

18. The BES according to claim 17, further comprising human cells of endothelial cell lineage that have been cultivated in vitro on the decellularized vessel matrix.

19. The BES according to claim 16, wherein the donor vessel is an allograft derived from a postmortem human vessel.

20. The BES according to claim 16, wherein the donor vessel is an isograft obtained from the body of the subject in need of the stent.

21. The BES according to claim 16, further comprising one or more drug-containing layers for attracting, differentiating, or proliferating cells of endothelial lineage.
